(19) United States
(12) Patent Application Publication
(10) Pub. No.: US 2020/0197864 A1
(43) Pub. Date: Jun. 25, 2020

(54) Method for Scavenging Airborne Formaldehyde

(71) Applicant: RHODIA OPERATIONS, Paris (FR)

(72) Inventors: Peng XU, (SG); Wei YUAN, (SG); Yanhui CHEN, Shanghai (CN); Julien Rabih RACHET, Shanghai (CN)

(21) Appl. No.: 16/613,294

(22) PCT Filed: May 15, 2018
(86) PCT No.: PCT/CN2018/086797

§ 371 Date: Nov. 13, 2019

(30) Foreign Application Priority Data
May 15, 2017 (CN) PCT/CN2017/084339

(51) Int. Cl. B01D 53/72; B01D 53/78; B01D 53/82; B01J 20/22; B01J 20/26; C07C 211/13; C07C 211/35

(52) U.S. Cl. CPC B01D 53/72; B01D 53/78; B01D 53/82; B01J 20/22; B01J 20/261; C07C 211/13; C07C 2601/14; B01D 2252/2041; B01D 2252/20421; B01D 2252/20426; B01D 2252/20431; B01D 2253/202; B01D 2257/704; C07C 211/35

(57) ABSTRACT

A method for scavenging airborne formaldehyde comprising a step of contacting the airborne formaldehyde with an amine compound or a salt thereof.

This application claims priority to PCT international patent application No. PCT/CN2017/084339 filed on May 15, 2017, the whole content of this application being incorporated herein by reference for all purposes.

### TECHNICAL FIELD

The present invention relates to a method for scavenging airborne formaldehyde. The method comprises a step of contacting the airborne formaldehyde with an amine compound.

### BACKGROUND ART

Formaldehyde is one of the most important starting materials in the chemical industry. It is used, for example, in the synthesis of urea formaldehyde resins, melamine resins, phenol formaldehyde resins, polyoxymethylene plastics and methylene diphenyl diisocyanates.

Formaldehyde is also a precursor to polyfunctional alcohols such as pentaerythritol, which is used to make paints and explosives. Other formaldehyde derivatives include methylene diphenyl diisocyanate, which is an important component in polyurethane paints and foams, and hexamine, which is used in phenol-formaldehyde resins.

In the textile industry use is made of formaldehyde-based resins as finishers to make fabrics crease-resistant. Formaldehyde is also used in the production of adhesives, textile aids, bonding agents, moulding sand binders, ion exchanger, moulded and extruded articles, casting resins and hardeners.

Due to the wide use of formaldehyde, formaldehyde containing material can be found in many products, such as those available for indoor use. One problem associated with the formaldehyde containing material is that it may emit formaldehyde to the environment because of the de-gassing of formaldehyde. Formaldehyde is classified as a probable human carcinogen by the U.S. Environmental Protection Agency and is thus harmful for human health. Such problem of formaldehyde containing material is in particular of concern when the material is placed in indoor environment. Hence, formaldehyde should be closely regulated to very low level in indoor environment.

Because of the aforementioned risks for human health, there is a strong need for effective scavengers for formaldehyde.

U.S. Pat. No. 3,255,150 discloses use of dithioxamides as formaldehyde scavenger. Those substances have the disadvantage in polymers that they decompose to sulphur, which accelerate cross-linking activities. Urea is also known as an inexpensive formaldehyde scavenger, but a disadvantage thereof is that scavenged formaldehyde is re-emitted by hydrolysis with heat.

There was a strong need for highly effective formaldehyde scavengers, which will be usable in various applications.

An object of the present invention therefore was to provide an effective formaldehyde scavenger.

### SUMMARY OF INVENTION

The present invention provides a method for scavenging airborne formaldehyde, comprising a step of contacting the airborne formaldehyde with an amine compound, wherein the amine compound has general formula (I):

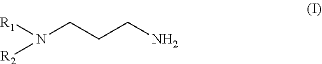

wherein R₁ and R₂ are independently hydrogen, a C₁-C₁₂ alkyl or hydroxyalkyl, a cycloalkyl or heterocycloalkyl such as a five or six membered cycloalkyl, an aryl or arylalkyl, or a group according to (-A-NR₃R₄);

preferably, at least one of R₁ and R₂ is not hydrogen; wherein A is a C₂-C₈ alkylene group such as ethylene and propylene, R₃ and R₄ are independently hydrogen, a C₁-C₁₂ alkyl or hydroxyalkyl, a cycloalkyl or heterocycloalkyl such as a five or six membered cycloalkyl, an aryl or arylalkyl, or a group according to —(CH₂)ₙ—NH₂, n is an integer of 1 to 12, preferably 2 to 6, in particular 2, 3 and 4;

preferably, at least one of R₃ and R₄ is not hydrogen.

The present invention further provides a method for scavenging airborne formaldehyde comprising a step of contacting the airborne formaldehyde with an amine compound, wherein the amine compound has general formula (II):

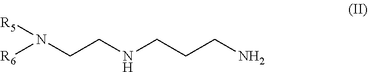

wherein R₅ and R₆ are independently hydrogen, C₁-C₁₂ alkyl or hydroxyalkyl, or a group according to —(CH₂)ₙ—NH₂; wherein n is an integer of 1 to 12, preferably 2 to 6, in particular 2, 3 and 4;

preferably, at least one of R₅ and R₆ is not hydrogen.

Alternatively, the amine compound according to the general formula (I) or the general formula (II) can be used in the form of salts. Suitable salts include hydrochloride, acetate, formate, oxalate, citrate, carbonate, sulfate, phosphate and salts of polyanions.

In one preferred embodiment, the present invention is directed to a method for scavenging airborne formaldehyde comprising a step of contacting the airborne formaldehyde with a polymeric composition which comprises the amine compound described herein or a salt of the amine compound.

### DETAILED DESCRIPTION

Throughout the description, including the claims, the term “comprising one” or “comprising a” should be understood as being synonymous with the term “comprising at least one”, unless otherwise specified, and “between” should be understood as being inclusive of the limits.

It should be noted that in specifying any range of concentration, weight ratio or amount, any particular upper concentration, weight ratio or amount can be associated with any particular lower concentration, weight ratio or amount, respectively.

The articles “a”, “an” and “the” are used to refer to one or to more than one (i.e., to at least one) of the grammatical object of the article.

The term “and/or” includes the meanings “and”, “or” and also all the other possible combinations of the elements connected to this term.

As used herein, the terminology “(Cₙ-Cₘ)” in reference to an organic group, wherein n and m are each integers, indicates that the group may contain from n carbon atoms to m carbon atoms per group.

“Alkyl”, as used herein, means a group or part of a group which refers to a straight or branched saturated aliphatic hydrocarbon group. Examples of suitable alkyl include methyl, ethyl, n-propyl, 2-propyl, n-butyl, sec-butyl, t-butyl, hexyl, and the like.

As used herein, the term “alkylene” refers to a saturated, straight-chain or branched-chain, divalent hydrocarbon radical. For example, methylene, ethylene, n-propylene, n-butylene, and the like.

As used herein, the term “cycloalkyl” refers to a saturated or partially saturated, monocyclic or fused or spiro polycyclic, carbocycle that may contain from 3 to 9 carbons per ring, such as cyclopropyl, cyclobutyl, cyclopentyl, cyclohexyl and the like, unless otherwise specified. It includes monocyclic systems such as cyclopropyl and cyclohexyl, bicyclic systems such as decalin, and polycyclic systems such as adamantane. The group may be a terminal group or a bridging group.

As used herein, “heterocycloalkyl” refers to a saturated or partially saturated monocyclic, bicyclic, or polycyclic ring containing at least one heteroatom selected from nitrogen, sulfur, oxygen. The heterocycloalkyl group may have from 1 to 3 heteroatoms in at least one ring. Each ring may be from 3 to 10 membered, such as 4 to 7 membered. Examples of suitable heterocycloalkyl substituents include pyrrolidyl, tetrahydrofuryl, tetrahydrothiofuranyl, piperidyl, piperazyl, tetrahydropyranyl, morphilino, 1,3-diazapane, 1,4-diazapane, 1,4-oxazepane, and 1,4-oxathiapane. The group may be a terminal group or a bridging group.

As used herein, the term “aryl” means a monovalent unsaturated hydrocarbon radical containing one or more six-membered carbon rings in which the unsaturation may be represented by three conjugated double bonds, which may be substituted one or more of carbons of the ring with hydroxy, alkyl, alkenyl, halo, haloalkyl, or amino, such as, for example, phenoxy, phenyl, methylphenyl, dimethylphenyl, trimethylphenyl, chlorophenyl, trichloromethylphenyl, aminophenyl.

“Arylalkyl”, as used herein, means an aryl-alkyl- group in which the aryl and alkyl moieties are as previously described. Preferred arylalkyl groups contain a C₁-C₅ alkyl moiety. Exemplary arylalkyl groups include benzyl, phenethyl and naphthelenemethyl. The group may be a terminal group or a bridging group.

The present invention provides a method for scavenging airborne formaldehyde by utilizing an amine compound or a salt thereof.

The amine compound has the general formula (I):

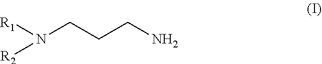

wherein R₁ and R₂ are independently hydrogen, a C₁-C₁₂ alkyl or hydroxyalkyl, a cycloalkyl or heterocycloalkyl, an aryl or arylalkyl, or a group according to (-A-NR₃R₄);

preferably, at least one of R₁ and R₂ is not hydrogen; wherein A is a C₂-C₈ alkylene group such as ethylene and propylene, R₃ and R₄ are independently hydrogen, a C₁-C₁₂ alkyl or hydroxyalkyl, a cycloalkyl or heterocycloalkyl such as a five or six membered cycloalkyl, an aryl or arylalkyl, or a group according to —(CH₂)ₙ—NH₂, n is an integer of 1 to 12, preferably 2 to 6, in particular 2, 3 and 4;

preferably, at least one of R₃ and R₄ is not hydrogen.

Advantageously, the amine compound has the general formula (I):

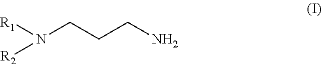

wherein R₁ and R₂ are independently hydrogen, a C₁-C₁₂ alkyl or hydroxyalkyl, a five or six membered cycloalkyl, an aryl or arylalkyl, or a group according to (-A-NR₃R₄);

at least one of R₁ and R₂ is not hydrogen;

wherein A is a C₂-C₈ alkylene group such as ethylene and propylene; R₃ and R₄ are independently hydrogen, a C₁-C₁₂ alkyl or hydroxyalkyl, a five or six membered cycloalkyl, an aryl or arylalkyl, or a group according to —(CH₂)ₙ—NH₂,

n is an integer of 1 to 12, preferably 2 to 6, in particular 2, 3 and 4.

Preferably, at least one of R₃ and R₄ as defined above is not hydrogen.

More advantageously, the amine compound has the general formula (II):

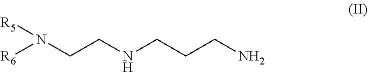

wherein R₅ and R₆ are independently hydrogen, C₁-C₁₂ alkyl or hydroxyalkyl, or a group according to —(CH₂)ₙ—NH₂;

wherein n is an integer of 1 to 12, preferably 2 to 6, in particular 2, 3 and 4.

Preferably, at least one of R₅ and R₆ as defined above is not hydrogen.

In one preferred embodiment of the present invention, the amine compound is selected from the group consisting of N,N′-bis-(3-aminopropyl)ethylenediamine, 3-cyclohexylaminopropylamine, N,N′-dimethyldipropylenetriamine, N,N,N′-tris(3-aminopropyl)ethylenediamine and a combination thereof.

It has been discovered that the amine compound of the present invention are capable to absorb airborne formaldehyde to remove free formaldehyde from the environment, namely an open or close space such as a room. Without wishing to be bound by theory, it is believed that this effect is due to the adduct formation between formaldehyde and the amine compound.

The present invention also provides a reaction product formed by reacting formaldehyde with the amine compound described herein.

It is appreciated that the amine compound may be used solely or in combination. The amine compound may also be used in combination with other compounds which can react with formaldehyde and scavenge it, such as urea. The amine compound may be used to scavenge airborne formaldehyde from the environment or airspace surrounding articles that emit formaldehyde. The amine compound may be used in various forms depending, for example, on the particular application and, in some instances, on the physical state of the amine compound or the resulting conjugate compound (i.e., whether liquid or solid). For instance, when the amine compound is a solid, it may be dispersed loosely, as in trays, or contained in a permeable container or cartridge that is placed in the formaldehyde contaminated environment. The amine compound may also, for example, be used as a powder formulation contained in a paper sachet similar to the type used for silica gel packages typically included in product packaging for moisture reduction. In this embodiment, sachets containing the amine compound may, for instance, be included in packaging to reduce build-up of formaldehyde levels in the packaging. When the amine compound is liquid, it may be absorbed onto a carrier, such as silica, wood powder and cotton, which is then placed in the formaldehyde contaminated environment.

In some aspects, the amine compound may be added in a formaldehyde-based composition. Incorporation of the amine compound can reduce airborne formaldehyde emitted from such formaldehyde-based composition.

More preferably, the amine compound may be added in a polymeric composition which does not emit formaldehyde or emits minimal formaldehyde, and such polymeric composition can be brought into contact with airborne formaldehyde for scavenging it. The polymeric composition, for example, may be a coating, a paint or an adhesive. The polymeric composition may also be a latex composition, which can be used as binder for formulating end products, such as coatings or adhesives.

For example, the amine compound may be added in a coating composition which is then coated directly onto a surface of an article that emits formaldehyde, such as a wood composite product, a packaging container used with such product, a building framework, or a fabric (e.g. drapery, carpet, air strip). Various coating methods can be employed depending on the substrate to be coated and include spraying, dipping, sizing, curtain coating, blade coating, and brush applications.

Alternatively, the coating composition incorporating the amine compound may be coated on an article which is close to the source of the airborne formaldehyde. For example, the coating composition may be coated on a wall, a floor, an article which is close to the source of the airborne formaldehyde in an indoor environment, such as in a room.

By way of further example, the polymeric composition incorporating the amine compound are suitable for use in warehouses, composite wood and textile production facilities, mobile homes, attics, areas of continual air flow such as air handling systems, air ducts, or near a heating/cooling system's intake or exhaust where the air flow would be concentrated. The polymeric composition can also be applied to substrates via extrusion into laminate layers, such as wallboard. The polymeric composition can also be added to carpet backsizings, deposited on wallpaper, included in non-formaldehyde glue e.g. wall paper paste, used in cabin filters to remove formaldehyde from incoming air, or in glass fiber insulation, via incorporation into/onto the facing paper, or coating on the fibers. The polymeric composition can also be used on textiles and fabrics such as permanent pressed products e.g. draperies, dress shirts etc. The polymeric composition can also be included in a consumer static air freshener module used in the home.

According to the present invention, the polymeric composition comprises at least a polymer, optionally further additives, pigments and/or solvents. Accordingly, the present invention also relates to a polymeric composition, such as a coating or adhesive composition, which comprises a polymer and the amine compounds described herein.

For example, the polymer may be an acrylic-based polymer, styrene-based polymer, vinyl-based polymer, alkyd, melamine, polyurethane or a mixture thereof.

The acrylic-based polymer may contain acrylic monomers, which include and are not limited to C₁-C₁₈ alkyl esters of (meth)acrylic acids, hydroxyl C₁-C₈ alkyl esters of (meth)acrylic acids, (meth)acrylic acids or acrylamide. Specific examples of acrylate monomers include methyl acrylate, ethyl acrylate, butyl acrylate, 2-ethyl hexyl acrylate, decyl acrylate, methyl methacrylate, butyl methacrylate, iso-butyl methacrylate, iso-bornyl methacrylate, hydroxy ethyl acrylate, hydroxy ethyl methacrylate and combination of these acrylate monomers.

The styrene-based polymer may contain vinyl aromatic monomers such as styrene, 1-vinyl napthalene, 2-vinyl napthalene, 3-methyl styrene, 4-propyl styrene, t-butyl styrene.

The vinyl-based polymer may contain monomers such as vinyl acetate, vinyl versatate, ethylene, vinyl chloride, acrolein, vinyl alkyl ketones having 4 to 7 carbon atoms such as vinyl methyl ketone, vinyl ethyl ketone or vinyl butyl ketone, N-vinyl caprolactam, N-vinyl pyrrolidone, 1,3-butadiene.

The polymer may also contain monomers such as γ-methacryloxypropyltris(2-methoxyethoxy)silane, vinylmethoxysilane, vinyltriethoxysilane, vinyldiethoxysilanol, vinylethoxysilanediol, allyltriethoxysilane, vinyltripropoxysilane, vinyltriisopropoxysilane, vinyltributoxysilane, vinyltriacetoxysilane, trimethylglycolvinylsilane, γ-methacryloxypropyltrimethylglycolsilane, γ-acryloxypropyltriethoxysilane and γ-methacryloxypropyltrimethoxysilane, cyclic ureido co-monomers.

The polymer may include, but is not limited to: all-acrylic copolymer, styrene-acrylic copolymer, vinyl acetate-acrylic copolymer, vinyl acetate-ethylene copolymer, polyvinyl acetate, polyvinyl acrylate, polyvinyl chloride, polyvinyl alcohol, polyvinyl ether, ethylene-vinyl chloride copolymer, butadiene styrene copolymer, N-vinyl caprolactam homopolymer, N-vinyl pyrrolidone copolymer, alkyd, melamine and polyurethane. Other similar polymers can also be used. Preferably, the polymer is all-acrylic copolymer, styrene-acrylic copolymer, vinyl acetate-acrylic copolymer or vinyl acetate-ethylene copolymer.

The polymeric composition may include and is not limited to: architectural coating, wood coating, auto coating, marine coating, floor coating, textile coating, coatings for plastics, pressure sensitive tape, sealing tape, plywood adhesive, tile adhesive, floor adhesive, paper adhesive, leather adhesive, laminate adhesive, and latex binder composition.

The polymeric composition incorporating the amine compound can be readily prepared by techniques well known in the art, for example the techniques disclosed in U.S. Pat. Nos. 5,500,246 and 6,025,449. The amine compound may be added to the polymeric composition and mixed with other components. Sequence of addition of the components is not particularly restricted. The amine compound may be pre-mixed with or added to a raw material that is used in the polymeric composition. For example, the amine compound may be added to a latex, which is used to make a final product such as a paint, coating or adhesive. Alternatively, the amine compound may be added during the formulation stage, at which pigments/additives are added to a base latex polymer to make it into a final product such as a paint, coating or adhesive. For example, in the case of preparing emulsion paints, the amine compound can be added either in the grinding stage or the letdown stage.

According to any one of the invention embodiments, the polymeric composition is preferably aqueous based, thus comprises water. More preferably, the polymeric composition is in the form of aqueous solution or dispersion.

The amine compound described herein can be used in the form of salts. Suitable salts include and are not limited to: hydrochloride, acetate, formate, oxalate, citrate, carbonate, sulfate, phosphate and salts of polyanions such as salts of polyglutamic acid, polyacrylic acid, 1,2,3,4-butanetetraacarboxylic acid.

Preferably, the polymeric composition comprises from 0.05 wt % to 10 wt % of the amine compound or a salt thereof based on the total weight of the composition, more preferably, from 0.1 wt % to 5 wt %, even more preferably, from 0.2 wt % to 2 wt %.

The polymeric composition may further comprise additives such as emulsifier, co-solvent, wetting agent, dispersant, pigment, extender, colorant, thickener, defoamer, levelling agent, coalescing agent, pH neutralizer, antioxidant, surfactant, biocide and combination thereof.

The polymeric composition may also be a solvent-based polymeric composition, in which case, the polymeric composition further comprises one or more organic solvents. Exemplary solvents include ketones, such as methyl ethyl ketone, methyl isobutyl ketone, methyl amyl ketone and diisobutyl ketone, esters such as butyl acetate andamyl acetate, aromatics such as toluene, xylenes and higher boiling mixtures of aromatics, aliphatics such as mineral spirits, odorless mineral spirits and VMP naphtha, and glycol ethers such as butyl ether of ethylene glycol and butyl ether of diethylene glycol. The polymers used in the solvent-based polymeric composition can be, but are not limited to: polyvinyl acetate, polyvinyl ether, polyester, polyacrylic, polychloroprene, polyisobutylene, polyvinyl butyral, acrylonitrile-butadiene, polyurethane, alkyd resin, phenolic resin, urea resin, melamine resin, epoxide resin. The solvent-based polymeric composition may contain additional components, such as cross-linking agents, defoamers, slip additives, wetting agents, pigments or colorants, waxes and the like.

The present invention is described in more detail by references to the following examples, without being limited to them.

Should the disclosure of any patents, patent applications, and publications which are incorporated herein by reference conflict with the description of the present application to the extent that it may render a term unclear, the present description shall take precedence.

### EXAMPLES

Materials

Thickener: Cellosize QP-30000 from the Dow Company

Wetting agent: Rhodoline® WA9 from the Solvay Company

Defoamers: Rhodoline® 681F, Rhodoline® 6002 and Rhodoline® 6008 from the Solvay Company

Dispersing agents: Rhodoline® 111, Rhodoline® DP270 from the Solvay Company

Dispersing agent: NUOSPERSE FX605 from the Elementis Specialties Company

Full-acrylic latex: Primal AC 261 latex from the Dow Company

Vinyl acetate/ethylene latex: EcoVAE 1608 latex from the Celanese Company

Associative thickener: Acrysol TT-935 from the Dow Company

Coalescing agent: Texanol from the Eastman Company pH neutralizer: AMP-95 from the Angus Chemical Company

Formaldehyde solution, 37 wt %, from Sigma-Aldrich

N,N′-bis-(3-aminopropyl)ethylenediamine (CAS 10563-26-5)

3-cyclohexylaminopropylamine (CAS 37221-91-3)

N,N′-dimethyldipropylenetriamine (CAS 10563-29-8)

N,N,N′-tris(3-aminopropyl)ethylenediamine (CAS 4963-47-7)

Polyacrylic acid, N,N,N′-tris(3-aminopropyl)ethylenediamine salt

Preparation of Coating Films

Coating films were prepared according to the formulations in Tables 1 and 2, using the following steps:

(1) Grind phase: Grind phase was prepared in a stainless steel beaker with a high-speed mixer having a 3.2-inch diameter Cowles-type blade. Firstly, water was added to the beaker, followed by other ingredients. Agitation speed was increased as the viscosity increased, to maintain a uniform mixture. Pigment and extenders were typically added at the last step. The final grind mixture was agitated at high speed (e.g. 1800 rpm) for a period of 20 min or more to accomplish disagglomeration of the primary pigment particles and formation of a stable dispersion. The fineness of grinds was determined by a hegman gauge. Value below 50 μm was considered acceptable.

(2) Letdown phase: Letdown phase was done by using the same equipment as described above. The ingredients were added into a vessel containing the grind mixture prepared in (1). Moderate shear (e.g. 800 rpm) was applied for a period of 20 min to activate thickeners and allow viscosity to develop. Final viscosity and pH of the fresh formulations were controlled at the value of 95-100 KU and 8.5-8.8, respectively.

(3) Addition of the formaldehyde scavengers (water was added in the Comparative Examples): Aliquots from stock paint prepared in (2) were transferred to another stainless steel beaker for preparing the final formulations. The amount of aliquot was actually weighted and the required amount of the formaldehyde scavengers or water was calculated. The formaldehyde scavengers or water were added under moderate shear using marine propeller blade at 800 rpm for 20 min.

(4) The obtained paints were then applied on a cast-coated food wrapping paper using a 150 μm applicator and dried under the controlled conditions of relative humidity (50%) and temperature (25° C.). After 7 days, the paint films were peeled off from the coated paper for formaldehyde scavenging efficiency measurement.

TABLE 1

| | | | | |
|---|---|---|---|---|
| EX1 | EX2 | EX3 | EX4 | CE1 |

| | |
|---|---|
| Components | Amount (g) |

Grind components

| | | | | | |
|---|---|---|---|---|---|
| Water | 129.5 | 129.5 | 129.5 | 129.5 | 129.5 |
| Propylene glycol | 10.0 | 10.0 | 10.0 | 10.0 | 10.0 |
| QP-30000 | 1.5 | 1.5 | 1.5 | 1.5 | 1.5 |
| AMP-95 | 0.5 | 0.5 | 0.5 | 0.5 | 0.5 |
| Rhodoline ® 111 | 7.0 | 7.0 | 7.0 | 7.0 | 7.0 |
| WA9 | 2.0 | 2.0 | 2.0 | 2.0 | 2.0 |
| Rhodoline ® 681F | 1.5 | 1.5 | 1.5 | 1.5 | 1.5 |
| TiCO₂ | 220.0 | 220.0 | 220.0 | 220.0 | 220.0 |
| CaCO₃ | 150.0 | 150.0 | 150.0 | 150.0 | 150.0 |
| Grind mix subtotal | 522.0 | 522.0 | 522.0 | 522.0 | 522.0 |

Letdown Components

| | | | | | |
|---|---|---|---|---|---|
| Rhodoline ® 681F | 0.5 | 0.5 | 0.5 | 0.5 | 0.5 |
| Primal AC 261 | 330.0 | 330.0 | 330.0 | 330.0 | 330.0 |
| Texanol | 16.0 | 16.0 | 16.0 | 16.0 | 16.0 |
| Acrysol TT-935 | 2.4 | 2.4 | 2.4 | 2.4 | 2.4 |
| Water | 124.1 | 124.1 | 124.1 | 124.1 | 124.1 |
| N,N′-bis-(3-aminopropyl) | 5.0 | — | — | — | — |
| ethylenediamine | | | | | |
| 3-cyclohexylamino- | — | 5.0 | — | — | — |
| propylamine | | | | | |
| N,N-Dimethyldipropyl- | — | — | 5.0 | — | — |
| enetriamine | | | | | |
| N,N,N′-tris(3- | — | — | — | 5.0 | — |
| aminopropyl)ethyl- | | | | | |
| enediamine | | | | | |
| Water | — | — | — | — | 5.0 |
| Total | 1000.0 | 1000.0 | 1000.0 | 1000.0 | 1000.0 |

(EX means Example and CE means Comparative Example)

TABLE 2

| | | | | |
|---|---|---|---|---|
| EX5 | EX6 | EX7 | EX8 | CE2 |

| | |
|---|---|
| Components | Amount (g) |

Grind components

| | | | | | |
|---|---|---|---|---|---|
| Water | 252.0 | 252.0 | 252.0 | 252.0 | 252.0 |
| QP-30000 | 4.0 | 4.0 | 4.0 | 4.0 | 4.0 |
| NUOSPERSE FX605 | 7.5 | 7.5 | 7.5 | 7.5 | 7.5 |
| AMP-95 | 2.0 | 2.0 | 2.0 | 2.0 | 2.0 |
| Rhodoline ® WA9 | 2.0 | 2.0 | 2.0 | 2.0 | 2.0 |
| Rhodoline ® DF6008 | 1.5 | 1.5 | 1.5 | 1.5 | 1.5 |
| TiCO₂ | 220.0 | 220.0 | 220.0 | 220.0 | 220.0 |
| Calcined Clay | 75.0 | 75.0 | 75.0 | 75.0 | 75.0 |
| CaCO₃ | 80.0 | 80.0 | 80.0 | 80.0 | 80.0 |
| Grind mix subtotal | 644.0 | 644.0 | 644.0 | 644.0 | 644.0 |

Letdown Components

| | | | | | |
|---|---|---|---|---|---|
| EcoVAE 1608 | 290.0 | 290.0 | 290.0 | 290.0 | 290.0 |
| Rhodoline ® DF 6008 | 1.5 | 1.5 | 1.5 | 1.5 | 1.5 |
| Acrysol TT-935 | 3.0 | 3.0 | 3.0 | 3.0 | 3.0 |
| Propylene Glycol | 15.0 | 15.0 | 15.0 | 15.0 | 15.0 |
| Water | 41.5 | 41.5 | 41.5 | 41.5 | 41.5 |
| N,N′-bis-(3-aminopropyl) | 5.0 | — | — | — | — |
| ethylenediamine | | | | | |
| 3-cyclohexylamino- | — | 5.0 | — | — | — |
| propylamine | | | | | |
| N,N-Dimethyldipropyl- | — | — | 5.0 | — | — |
| enetriamine | | | | | |
| N,N,N′-tris(3- | — | — | — | 5.0 | — |
| aminopropyl)ethyl- | | | | | |
| enediamine | | | | | |
| Water | — | — | — | — | 5.0 |
| Total | 1000.0 | 1000.0 | 1000.0 | 1000.0 | 1000.0 |

(EX means Example and CE means Comparative Example)

Formaldehyde-Scavenging Efficiency Measurement

The efficiency in lowering airborne formaldehyde was measured by using Headspace Gas Chromatography/Mass Spectrometry (HS-GCMS). The paint film (0.01 g) was added into a 15 mL standard HS-GCMS bottle. 5 μL of formaldehyde solution (20,000 ppm) which was contained in a 1.5 mL plastic centrifuge tube, were then put into the bottle. The bottle was sealed for 1 day under controlled conditions of relative humidity (50%) and temperature (25° C.). The paint films were expected to absorb or react with the formaldehyde in the bottle. The formaldehyde gases remaining in the bottle were injected into GCMS to evaluate the efficiency of the formaldehyde scavengers in reducing airborne formaldehyde. The remaining formaldehyde amount was measured by HS-GCMS at 40° C. and 100° C., respectively (the incubator was set at 40° C. for 3 min and 100° C. for 3 min). The area of formaldehyde peak in HS-GCMS chromatogram was measured, which corresponds to the amount of remaining formaldehyde.

Scavenging efficiency was calculated by the following equation: Scavenging efficiency (%)=(F_{Total}−Fₛ)/F_{Total}*100 F_{Total} represents the peak area of formaldehyde determined in the bottle without any paint film; Fₛ represents the peak area of formaldehyde determined in the bottle with paint film in which the formaldehyde scavenger or water is applied.

Scavenging efficiency results are shown in Table 3 below:

TABLE 3

Scavenging Efficiency

| | at 40° C. | at 100° C. |
|---|---|---|
| Example 1 | 65.67 | 54.22 |
| Example 2 | 46.20 | 21.65 |
| Example 3 | 47.12 | 20.94 |
| Example 4 | 64.44 | 57.52 |
| Comparative Example 1 | 25.48 | 1.37 |
| Example 5 | 59.61 | 58.91 |
| Example 6 | 34.92 | 27.65 |
| Example 7 | 40.41 | 24.75 |
| Example 8 | 60.26 | 55.45 |
| Comparative Example 2 | 23.29 | 3.22 |

As shown in Table 3, the inventive amine compounds (N,N′-bis-(3-aminopropyl)ethylenediamine, 3-cyclohexylaminopropylamine, N,N-dimethyldipropylenetriamine and N,N,N′-tris(3-aminopropyl)ethylenediamine) effectively lowered airborne formaldehyde levels at both 40° C. and 100° C. Paint films without the formaldehyde scavengers also showed some efficiency (25.48% and 23.29%) at 40° C., however, the efficiency was markedly lower at 100° C. (1.37% and 3.22%). Such significant reduction in efficiency at increased temperature indicated that the paint films without the formaldehyde scavengers lowered airborne formaldehyde mainly by physical absorption and the physically-absorbed formaldehyde was re-emitted when the temperature was increased. In contrast, coating films with the inventive amine compounds showed satisfactory efficiency in reducing airborne formaldehyde level at both 40° C. and 100° C., which indicated that they could chemically react with the formaldehyde and decomposition was minimal even at a higher temperature.

Another set of experiments were conducted according to the procedure as described above. The formulation tested and the scavenging efficiency results are shown in Tables 4 and 5 below, respectively:

TABLE 4

| | |
|---|---|
| | Comparative |
| Example 9 | Example 3 |

| | |
|---|---|
| Components | Amount (g) |

Grind components

| | | |
|---|---|---|
| Water | 265.1 | 265.1 |
| QP-30000 | 4.0 | 4.0 |
| AMP-95 | 0 | 2.42 |
| Rhodoline ® DP270 | 0 | 9.52 |
| Polyacrylic acid, N,N,N′-tris(3- | 19 | 0 |
| aminopropyl)ethylenediamine salt | | |
| Rhodoline ® WA9 | 2.0 | 2.0 |
| Rhodoline ® DF6002 | 1.05 | 1.05 |
| TiCO₂ | 220.0 | 220.0 |
| Calcined Clay | 75.0 | 75.0 |
| CaCO₃ | 80.0 | 80.0 |
| Grind mix subtotal | 666.13 | 659.07 |

Letdown Components

| | | |
|---|---|---|
| EcoVAE 1608 | 290.0 | 290.0 |
| Rhodoline ® DF 6002 | 1.05 | 1.05 |
| Acrysol TT-935 | 1.13 | 1.98 |
| Propylene Glycol | 15.0 | 15.0 |
| Water | 26.69 | 32.9 |
| Total | 1000.0 | 1000.0 |

TABLE 5

Scavenging Efficiency

| | at 40° C. | at 100° C. |
|---|---|---|
| Example 9 | 54.09 | 57.37 |
| Comparative Example 3 | 8.93 | 9.81 |

Results showed that the inventive formaldehyde scavenger effectively lowered airborne formaldehyde.

1. A method for scavenging airborne formaldehyde, comprising a step of contacting the airborne formaldehyde with an amine compound or a salt thereof, wherein the amine compound has general formula (I): 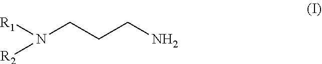
wherein R₁ and R₂ are independently hydrogen, a C₁-C₁₂ alkyl or hydroxyalkyl, a cycloalkyl or heterocycloalkyl, an aryl or arylalkyl, or a group according to (-A-NR₃R₄);
wherein A is a C₂-C₈ alkylene group, R₃ and R₄ are independently hydrogen, a C₁-C₁₂ alkyl or hydroxyalkyl, a cycloalkyl or heterocycloalkyl, an aryl or arylalkyl, or a group according to —(CH₂)ₙ—NH₂, n is an integer of 1 to 12.

2. The method according to claim 1, wherein the method comprises a step of contacting the airborne formaldehyde with an amine compound, the amine compound has general formula (I): 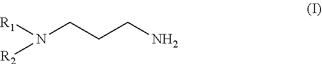
wherein R₁ and R₂ are independently hydrogen, a C₁-C₁₂ alkyl or hydroxyalkyl, a cycloalkyl or heterocycloalkyl, an aryl or arylalkyl, or a group according to (-A-NR₃R₄);
wherein A is a C₂-C₈ alkylene group, R₃ and R₄ are independently hydrogen, a C₁-C₁₂ alkyl or hydroxyalkyl, a cycloalkyl or heterocycloalkyl, an aryl or arylalkyl, or a group according to —(CH₂)ₙ—NH₂, n is an integer of 1 to 12.

3. The method according to claim 1 or 2, wherein the amine compound has the general formula (I): 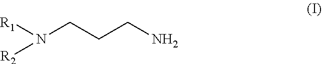
wherein R₁ and R₂ are independently hydrogen, a C₁-C₁₂ alkyl or hydroxyalkyl, a five or six membered cycloalkyl, an aryl or arylalkyl, or a group according to (-A-NR₃R₄);
wherein A is a C₂-C₈ alkylene group, R₃ and R₄ are independently hydrogen, a C₁-C₁₂ alkyl or hydroxyalkyl, a five or six membered cycloalkyl, an aryl or arylalkyl, or a group according to —(CH₂)ₙ—NH₂, n is an integer of 1 to 12.

4. The method according to any one of claim 1 to 3, wherein at least one of R₁ and R₂ as defined in the general formula (I) is not hydrogen.

5. The method according to any one of claims 1 to 4, wherein the amine compound has the general formula (II): 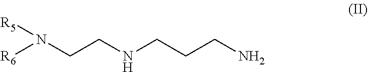
wherein R₅ and R₆ are independently hydrogen, C₁-C₁₂ alkyl or hydroxyalkyl, or a group according to —(CH₂)ₙ—NH₂; wherein n is an integer of 1 to 12.

6. The method according to claim 5, wherein at least one of R₅ and R₆ as defined in the general formula (II) is not hydrogen.

7. The method according to any one of claims 1 to 6, wherein the amine compound is selected from the group consisting of N,N′-bis-(3-aminopropyl)ethylenediamine, 3-cyclohexylaminopropylamine, N,N-dimethyldipropylenetriamine, N,N,N′-tris(3-aminopropyl)ethylenediamine and a combination thereof.

8. The method according to any one of claims 1 to 7, wherein the method comprises a step of contacting the airborne formaldehyde with a polymeric composition which comprises the amine compound or a salt thereof.

9. The method according to claim 8, wherein the polymeric composition further comprises a polymer.

10. The method according to claim 9, wherein the amine compound or the salt thereof is present in an amount of 0.05 wt % to 10 wt % based on total weight of the polymeric composition.

11. The method according to claim 9, wherein the amine compound or the salt thereof is present in an amount of 0.1 wt % to 5 wt % based on total weight of the polymeric composition.

12. A polymeric composition comprising:
(a) an amine compound of general formula (I) or a salt thereof: 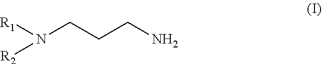
wherein R₁ and R₂ are independently hydrogen, a C₁-C₁₂ alkyl or hydroxyalkyl, a cycloalkyl or heterocycloalkyl, an aryl or arylalkyl, or a group according to (-A-NR₃R₄);
wherein A is a C₂-C₈ alkylene group, R₃ and R₄ are independently hydrogen, a C₁-C₁₂ alkyl or hydroxyalkyl, a cycloalkyl or heterocycloalkyl, an aryl or arylalkyl, or a group according to —(CH₂)ₙ—NH₂, n is an integer of 1 to 12; and
(b) a polymer selected from the group consisting of acrylic-based polymer, styrene-based polymer, vinyl-based polymer, alkyd, melamine, polyurethane and a mixture thereof.

13. The polymeric composition according to claim 12, wherein the polymer is selected from the group consisting of: all-acrylic copolymer, styrene-acrylic copolymer, vinyl acetate-acrylic copolymer, vinyl acetate-ethylene copolymer, polyvinyl acetate, polyvinyl acrylate, polyvinyl chloride, polyvinyl alcohol, polyvinyl ether, ethylene-vinyl chloride copolymer, butadiene styrene copolymer, N-vinyl caprolactam homopolymer and N-vinyl pyrrolidone copolymer.

14. The polymeric composition according to claim 12 or 13, wherein the polymer is selected from the group consisting of all-acrylic copolymer, styrene-acrylic copolymer, vinyl acetate-acrylic copolymer and vinyl acetate-ethylene copolymer.

15. A reaction product formed by reacting formaldehyde with an amine compound of general formula (I): 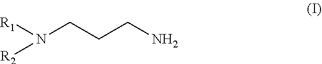
wherein R₁ and R₂ are independently hydrogen, a C₁-C₁₂ alkyl or hydroxyalkyl, a cycloalkyl or heterocycloalkyl, an aryl or arylalkyl, or a group according to (-A-NR₃R₄);
wherein A is a C₂-C₈ alkylene group, R₃ and R₄ are independently hydrogen, a C₁-C₁₂ alkyl or hydroxyalkyl, a cycloalkyl or heterocycloalkyl, an aryl or arylalkyl, or a group according to —(CH₂)ₙ—NH₂, n is an integer of 1 to 12.

16. Use of an amine compound of the general formula (I) or a salt thereof as a formaldehyde scavenger, 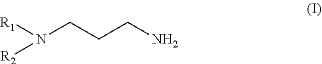
wherein R₁ and R₂ are independently hydrogen, a C₁-C₁₂ alkyl or hydroxyalkyl, a cycloalkyl or heterocycloalkyl, an aryl or arylalkyl, or a group according to (-A-NR₃R₄);
wherein A is a C₂-C₈ alkylene group, R₃ and R₄ are independently hydrogen, a C₁-C₁₂ alkyl or hydroxyalkyl, a cycloalkyl or heterocycloalkyl, an aryl or arylalkyl, or a group according to —(CH₂)ₙ—NH₂, n is an integer of 1 to 12.

17. The use according to claim 16, wherein the amine compound has the general formula (I): 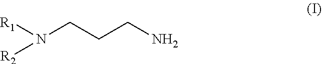
wherein R₁ and R₂ are independently hydrogen, a C₁-C₁₂ alkyl or hydroxyalkyl, a five or six membered cycloalkyl, an aryl or arylalkyl, or a group according to (-A-NR₃R₄);
wherein A is a C₂-C₈ alkylene group, R₃ and R₄ are independently hydrogen, a C₁-C₁₂ alkyl or hydroxyalkyl, a five or six membered cycloalkyl, an aryl or arylalkyl, or a group according to —(CH₂)ₙ—NH₂, n is an integer of 1 to 12.

18. The use according to claim 16 or 17, wherein the amine compound has the general formula (II): 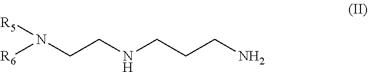
wherein R₅ and R₆ are independently hydrogen, C₁-C₁₂ alkyl or hydroxyalkyl, or a group according to —(CH₂)ₙ—NH₂; wherein n is an integer of 1 to 12.
